(19) United States
(12) Patent Application Publication
(10) Pub. No.: US 2006/0147530 A1
(43) Pub. Date: Jul. 6, 2006

(54) Sustained release compositions containing alfuzosin

(76) Inventors: Narayanan Badri Viswanathan, Madipakkam (IN); Sankar Ramakrishnan, Thajavur districk (IN); Rajeev Singh Raghuvanshi, New Delhi (IN); Ashok Rampal, Amritsar (IN)

Correspondence Address: RANBAXY INC., 600 COLLEGE ROAD EAST, SUITE 2100, PRINCETON, NJ, 08540, US

(21) Appl. No.: 10/532,296

(22) PCT Filed: Oct. 22, 2003
(86) PCT No.: PCT/IB03/04677

§ 371 Date: Feb. 6, 2006

(30) Foreign Application Priority Data
Oct. 22, 2002 (IN) 1023/DEL/2002

(51) Int. Cl. A61K 9/26

(52) U.S. Cl. 424/470

(57) ABSTRACT

The present invention relates to pharmaceutical compositions of alfuzosin or pharmaceutically acceptable salt, solvate, enantiomers or mixtures thereof, that release the active ingredient over an extended period of time. The pharmaceutical composition can be a sustained release oral dosage form that includes a single functional layer and, optionally, one or more nonfunctional layers adjacent to the single functional layer. The single functional layer includes alfuzosin or pharmaceutically acceptable salt, solvate, enantiomers or mixtures thereof and one or more release retarding ingredients.

### FIELD OF THE INVENTION

The present invention relates to pharmaceutical compositions of alfuzosin or pharmaceutically acceptable salt, solvate, enantiomers or mixtures thereof, that release the active ingredient over an extended period of time.

### BACKGROUND OF THE INVENTION

Alfuzosin is a selective alpha-1 adrenoceptor antagonist that belongs to the chemical class of 4-amino-6,7-dimethoxy quinazol-2-yl-alkylene diamines. Alfuzosin acts as a selective and competitive antagonist of alpha-1 adrenoceptor mediated contraction of prostatic, prostatic capsule, bladder base and proximal urethral structures and is used in the treatment of symptoms of benign prostatic hyperplasia.

Alfuzosin has a short half-life and shows the characteristic of being absorbed preferentially in the upper part of the gastrointestinal tract and, in particular, being absorbed in the duodenum and the jejunum. Sustained release compositions of alfuzosin provide various advantages over conventional multiple dosing including better patient compliance, reduced fluctuations of plasma drug levels, and reduced toxicity.

Alfuzosin is marketed exclusively for the treatment of benign prostatic hyperplasia and, more specifically, for the treatment of the symptoms associated with benign prostatic hyperplasia. Alfuzosin is indicated for the treatment of moderate to sever symptoms of benign prostatic hyperplasia.

Amongst the various dosage forms that have been submitted and approved by regulatory agencies in Europe, the USA, and other countries there are various administration regimens. For example, the 2.5 mg immediate release tablet dosage form is generally administered three times per day. The 5 mg modified release tablet may be administered once or twice per day depending on the age of the patient and the condition to be treated. A once daily formulation of alfuzosin, Xatral-XL (available in Europe) and UroXatral (recently approved in the USA), provides equivalent systemic exposure when compared to the 2.5 mg immediate release tablet dosage form of alfuzosin administered thrice daily. These once daily formulations were developed to provide a controlled release of alfuzosin over an extended period of time for 24 hours.

The alfuzosin 5 mg extended release dosage form may be given to adults twice daily, with the first dose taken at bedtime. The dose for elderly patients may be one 5 mg extended release tablet per day, taken at bedtime. This dosage can be increased to 10 mg per day, given as a single extended release 5 mg tablet taken twice daily.

U.S. Pat. No. 6,149,940 discloses a preparation of an alfuzosin 10 mg once daily composition for oral delivery using a technology termed Geomatrix that has been developed by Jagotec-AG. The three-layer Geomatrix tablet described in the '940 patent consists of a hydrophilic active matrix core containing alfuzosin hydrochloride and two inert, functional layers (one swellable layer and one erodible layer) whose functions are to control the hydration and swelling rate of the core, and thereby slow down and linearize the dissolution of the drug. When the tablet comes into contact with gastric juices, it increases considerably in volume and thus remains in the stomach for a longer time. In this manner, most of the drug is absorbed in a controlled manner in the portion of the gastrointestinal tract having the highest capacity for absorption. The alfuzosin is released in zero order from the dosage form developed using this technology. However, the manufacture of multi-layered tablets by this technology involves special facilities, is time consuming, complex to produce, and consequently relatively expensive.

U.S. Pat. No. 5,589,190 discloses a pharmaceutical composition that includes an alfuzosin core. The core is coated with a coating whose dissolution is pH dependent, which thereby enables the release of alfuzosin to be modulated over the entire length of the digestive tract. The '190 patent teaches that the sustained release of alfuzosin is dependent on the nature and thickness of the coating. Further, the '190 patent discloses a combination of two types of tablets with different release rates that are filled into hard gelatin capsules for once-daily oral administration. These coated formulations, however, have disadvantages including the possibility of leakage of active ingredient from the coating and the need for strict process controls during their manufacture.

EP700285 discloses drug delivery compositions of alpha adrenoceptor blocking agents that have a biphasic drug release profile. This patent teaches matrix compositions using hydroxypropyl methylcellulose and a coating that is designed to dissolve under the conditions present in the colonic region.

U.S. Pat. No. 4,259,314 discloses a dry pharmaceutical formulation containing a therapeutic agent and a dry carrier that includes hydroxypropyl methylcellulose and hydroxypropyl cellulose. This patent is directed towards the use of formulations with hygroscopic active ingredients.

U.S. Pat. No. 4,704,285 discloses the use of a fine particle size hydroxypropyl cellulose either alone or blended with hydroxypropyl methylcellulose for sustained release applications. However, this patent is not directed to any specific active ingredient and it involves the use of a specific grade of hydroxypropyl cellulose with specific particle size.

U.S. Pat. No. 4,680,323 discloses a sustained release pharmaceutical formulation used to administer active ingredients over a 12 to 24 hour time frame. The formulation includes a carrier prepared from hydroxypropyl methylcellulose, hydroxypropyl cellulose and a carboxyvinyl polymer. This patent teaches that carboxyvinyl polymer is a weak acid that reacts to form salts and thereby provide a zero order release rate under the alkaline conditions found in the small intestine.

EP0413061 discloses a sustained release formulation containing an active ingredient and a combination of hydroxypropylmethylcellulose and hydroxypropylcellulose. The hydroxypropylmethylcellulose used in the formulation is selected from two different molecular weights that range from 30,000 to 350,000 and 9,000 to 30,000, respectively. The hydroxypropylcellulose used in the formulation has a hydroxypropoxy content of 7 wt % to 16 wt %. This patent further teaches the use of combinations of at least three cellulose based polymers.

### SUMMARY OF THE INVENTION

In one aspect there is provided a sustained release oral dosage form that includes a single functional layer and, optionally, one or more nonfunctional layers adjacent to the single functional layer. The single functional layer includes alfuzosin or pharmaceutically acceptable salt, solvate, enantiomers or mixtures thereof and one or more release retarding ingredients.

Embodiments of the sustained release oral dosage form may include one or more of the following features. For example, the release retarding ingredient may be one or more of cellulose polymer, methacrylate polymer, acrylic acid polymer, block copolymer, gum and polyethylene oxide. The cellulose polymer may be one or more of hydroxypropyl methylcellulose, methylcellulose, hydroxypropylethylcellulose and hydroxypropyl cellulose. The gum may be one or more of xanthan gum, alginic acid, sodium alginate and locust bean gum.

The single functional layer may further include one or more pharmaceutically acceptable excipients. The one or more pharmaceutically acceptable excipients may include one or more of binders, diluents, and lubricants/glidants. The binder may be one or more of polyvinyl pyrrolidone, pregelatinized starch, and gelatin. The diluent may be one or more of lactose, mannitol, and microcrystalline cellulose. The lubricant may be one or more of magnesium stearate, zinc stearate, talc, and colloidal silicon dioxide.

The functional layer may be between about 10% to about 90% w/w of hydroxypropyl methylcellulose and between about 10% to about 90% w/w of hydroxypropyl cellulose. The functional layer may be between about 10% to about 70% w/w of hydroxypropyl methylcellulose, between about 10% to about 70% w/w of hydroxypropyl cellulose and between about 1% to about 20% w/w of methacrylic acid copolymer. The functional layer may be between about 10% to about 70% w/w of hydroxypropyl methylcellulose, between about 10% to about 70% w/w of hydroxypropyl cellulose, between about 5% to about 10% w/w of methacrylic acid copolymer, and between about 10% to about 50% w/w of lactose.

The sustained release dosage form may be in the form of one or more of tablets, capsules, pellets, granules and other dosage forms suitable for oral administration.

The sustained release oral dosage form may have a dissolution of less than about 17% in about 1 hour, less than about 61% in about 8 hours, less than about 94% in about 20 hours, as measured in a pH 6.8 phosphate buffer using USP Type II apparatus with a paddle speed of 100 rpm, at 37+/−2° C.

The sustained release oral dosage form may have a dissolution of less than about 26% in about 2 hours, less than about 77% in about 12 hours, and less than about 96% in about 24 hours, as measured in a pH 6.8 phosphate buffer using USP Type II apparatus with a paddle speed of 100 rpm, at 37+/−2° C.

The sustained release oral dosage form may have a dissolution of less than about 39% in about 4 hours and less than about 88% in about 16 hours, as measured in a pH 6.8 phosphate buffer using USP Type II apparatus with a paddle speed of 100 rpm, at 37+/−2° C.

The single functional layer may include granules. The one or more nonfunctional layers adjacent to the single functional layer may include a cosmetic coating. The cosmetic coating may include a colorant.

In another general aspect there is provided a method of treating secondary symptoms associated with benign prostatic hyperplasia. The method of treating includes administering a sustained release oral dosage form that includes a single functional layer and, optionally, one or more nonfunctional layers adjacent to the single functional layer. The single functional layer includes alfuzosin or pharmaceutically acceptable salt, solvate, enantiomers or mixtures thereof and one or more release retarding ingredients.

Embodiments of the method of treating may include one or more of the following features and/or those described above. For example, the release retarding ingredient may include one or more of cellulose polymer, methacrylate polymer, acrylic acid polymer, block copolymer, gum and polyethylene oxide. The cellulose polymer may be one or more of hydroxypropyl methylcellulose, methylcellulose, hydroxypropylethylcellulose and hydroxypropyl cellulose. The gum may be one or more of xanthan gum, alginic acid, sodium alginate and locust bean gum.

The sustained release oral dosage form may be administered either twice daily or once daily.

The single functional layer may include granules. The one or more nonfunctional layers adjacent to the single functional layer may include a cosmetic coating. The cosmetic coating may include a colorant.

In another general aspect there is provided a process for forming a sustained release oral dosage form. The process includes:

forming a mixture of alfuzosin or pharmaceutically acceptable salt, solvate, enantiomers or mixtures thereof and one or more release retarding ingredients;

forming a dosage form having a single functional layer from the mixture; and

optionally forming one or more nonfunctional layers adjacent to the single functional layer.

Embodiments of the process may include one or more of the following features and/or those described above. For example, the release retarding ingredient may be one or more of cellulose polymer, methacrylate polymer, acrylic acid polymer, block copolymer, gum and polyethylene oxide. The cellulose polymer may be one or more of hydroxypropyl methylcellulose, methylcellulose, hydroxypropyl ethylcellulose and hydroxypropyl cellulose. The gum may be one or more of xanthan gum, alginic acid, sodium alginate and locust bean gum.

The one or more nonfunctional layers adjacent to the single functional layer may be a cosmetic coating. The cosmetic coating may include a colorant.

In the process, forming a mixture may include one or more of direct compression, wet granulation, and dry granulation. Forming a mixture may include forming granules that include the alfuzosin or pharmaceutically acceptable salt, solvate, enantiomers or mixtures thereof and one or more release retarding ingredients.

Forming a mixture may further include adding one or more pharmaceutically acceptable excipients to the mixture.

Forming the dosage form having a single functional layer may include one or more of forming tablets, capsules, pellets, granules or other dosage forms suitable for oral administration. Forming the dosage form may further include compressing to form a tablet or filling into a capsule.

The details of one or more embodiments of the invention are set forth in the description below. Other features, objects, and advantages of the invention will be apparent from the description and the claims.

### DETAILED DESCRIPTION OF THE INVENTION

The inventors have recognized that there is an unmet and unrecognized need for a simpler sustained release dosage form of alfuzosin. In particular, the inventors have now discovered that a sustained release formulation of alfuzosin can be effectively made in the form of a dosage form having a single functional layer that includes alfuzosin or its salts, solvates, hydrates, enantiomers, or mixture thereof, a release retarding ingredient, and one or more pharmaceutically acceptable excipients. The dosage form may include one or more optional nonfunctional layers adjacent to the function layer.

The matrix composition of the present invention may include the active ingredient in a range of about 1 mg to about 30 mg. Two preferred dosage forms contain either 5 mg or 10 mg of active ingredient. The term “active ingredient” as used herein refers to alfuzosin or its salt, solvate, hydrates, enantiomer or mixtures thereof.

The term “release retarding ingredient” as used herein refers to any suitable polymer capable of retarding the release of active ingredient for about 12 to about 24 hours. Suitable release retarding ingredients include one or more of cellulose derivatives, acrylic acid or methacrylate polymers/copolymers, gums, vinyl alcohol or vinylpyrrolidone based polymers, block copolymers, polyethylene oxide, lipids and waxes. Suitable cellulose polymers include, for example, one or more of hydroxypropyl methylcellulose, hydroxypropyl cellulose, hydroxypropyl ethylcellulose, hydroxyethylcellulose, carboxymethylcellulose and methylcellulose. Suitable gums include, for example, one or more of xanthum gum, caraya gum, locust bean gum, alginic acid, and sodium alginate. The acrylic acid or methacrylic acid/methacrylate based polymers may include one or more of Eudragit polymers, such as Eudragit L-100, L 30 D-55, L-100 55, S-100. Suitable waxes include paraffin, carnauba, beeswax, or equivalents. Suitable lipids include hydrogenated vegetable oil, long chain fatty acid esters, and derivatives thereof.

Preferred cellulose polymers include hydroxypropyl methylcellulose 2208 with a molecular weight of 3,000-150,000, which is available from Dow Chemical Co. under the brand names Methocel K100M CR and Methocel K15M CR. Another preferred cellulose polymer is hydroxypropyl cellulose available under the brand names HPC from Nippon Soda Co. and Klucel from Aqualon. A preferred methacrylic polymer is Eudragit L 100 55. A preferred filler is lactose DCL-11. A preferred binder is polyvinylpyrrolidone K-30.

The amount of release retarding ingredient in the composition ranges from about 10% to about 90%, preferably between about 30% to about 80%, and more preferably between about 50% to about 75% by weight of the composition.

The amount of lubricants/glidants in the composition ranges from about 0.5% to about 5%, and more preferably between about 1.0% to about 3.0% by weight of the composition. The amount of binders in the composition ranges from about 2% to about 10%, and more preferably about 3.5% to 6%. The amount of filler varies from about 10% to about 60%, and more preferably from about 12% to about 30%. These amounts (i.e., release retarding ingredient, lubricant/glidant, and binder) are based on the weight of the composition.

The pharmaceutically acceptable excipients may be selected, for example, from binders such as polyvinyl pyrrolidone, pregelatinized starch and gelatin; diluents such as lactose, mannitol and microcrystalline cellulose; and lubricants/glidants such as magnesium stearate, zinc stearate, talc and colloidal silicon dioxide.

In one preferred embodiment, the sustained release dosage form includes hydroxypropyl methylcellulose in amounts ranging from about 10% to about 90% w/w, hydroxypropyl methylcellulose in amounts ranging from about 15% to about 50% w/w, hydroxypropyl cellulose in amounts ranging from about 10% to about 90% w/w, hydroxypropyl cellulose in amounts ranging from about 15% to 50% w/w, Eudragit L-100 55 in amounts ranging from about 1% to about 20% w/w, Eudragit L-100 55 in amounts ranging from about 4% to about 12% w/w, lactose in amounts ranging from about 10% to 7 about 60% w/w, polyvinyl pyrrolidone in amounts ranging from about 2% to about 10% w/w, magnesium stearate in amounts ranging from about 0.1% to about 5% W/w, talc in amounts ranging from about 0.1% to about 5% w/w, and colloidal silicon dioxide in amounts ranging from about 0.1% to 5% w/w.

The sustained release composition may be ultimately processed in the form of tablets, capsules, pellets, granules or other dosage form suitable for oral administration. The tablets may be prepared by various techniques such as direct compression, wet granulation or dry granulation. The tablets may be optionally coated with a nonfunctional coating to form a nonfunctional layer. The tablet/minitablets may be optionally filed into capsules.

“Cₘₐₓ,” as used herein, means the maximum plasma concentration of the active ingredient, produced by the ingestion of the composition of the invention or the reference product. “Tₘₐₓ,” as used herein, means the time to the maximum observed plasma concentration. “AUC” as used herein, means the area under the plasma concentration-time curve over the specified time interval for all the compositions.

The term “reference product” as used herein refers to the formulations containing alfuzosin or its salt, solvate, enantiomers or mixtures thereof, which release alfuzosin for an extended period of time of about 12 hours or about 24 hours, more preferably are prepared by Geomatrix technology and marketed in various countries. For example, the reference product may be the 5 mg and 10 mg Xatral-XL available in Europe, or the 10 mg UroXatral available in USA.

The term “substantially equivalent” as used in this specification and the appended claims refers to achieving a ratio (composition of present invention/reference product, e.g., Xatral-XL or UroXatral) of Cₘₐₓ and AUC_{0-inf} in the range of 80% to 125%.

The following examples are provided to further exemplify the invention, and are not intended to limit the scope of the invention.

### Example 1

TABLE 1
Formulation of Example 1

| S. No. | Ingredient | mg/tab | % w/w |
|---|---|---|---|
| 1 | Alfuzosin Hydrochloride | 10 | 2.86 |
| 2 | Hydroxypropyl methyl cellulose | 85 | 24.29 |
| | (Methocel K100M CR) | | |
| 3 | Hydroxypropyl cellulose (M) | 155 | 44.29 |
| 4 | Lactose | 77 | 22.0 |
| 5 | Polyvinyl pyrrolidone (PVP K30) | 15 | 4.29 |
| 6 | Talc | 2 | 0.57 |
| 7 | Colloidal silicon dioxide | 2 | 0.57 |
| 8 | Magnesium Stearate | 2 + 2 | 1.14 |
| | (intragranular and extragranular) | | |
| | Total Core Tablet Weight | 350 | |
| | Coating OPADRY White | 2.5% | |
| | (non-functional dressing) | | |

Alfuzosin, colloidal silicon dioxide and an approximately equal quantity of lactose were mixed and sifted through an American Society for Testing and Materials (ASTM) #60 mesh. The sifted material was geometrically diluted with sifted (ASTM #60 mesh) lactose until all the lactose had been added. This mixture was sifted again through an ASTM #60 mesh to increase homogeneity. To the above mixture, sifted (ASTM #30 mesh) hydroxypropyl methylcellulose, hydroxypropyl cellulose and polyvinyl pyrrolidone was added by geometric dilution technique. The resulting mixture was lubricated with talc and the intragranular portion of magnesium stearate. This resulting mixture was compacted in a roll-compactor and milled to obtain granules of less than ASTM #25. The granules thus obtained were lubricated with extragranular magnesium stearate portion and compressed into tablets using round punch toolings. The resulting tablets were coated with OPADRY of white color using a dispersion in isopropyl alcohol-water (50:50 mixture) to a weight build up of about 2.5%. Studies were conducted of the drug release profile of these tablets in 0.01N HCl in one test and pH 6.8 phosphate buffer in a second test using a USP Type II apparatus with a paddle speed of 100 rpm, at 37±2° C. The results of these studies are shown in Table 2.

TABLE 2
Dissolution Profile of the Formulation of Example 1

Percent released

| Time (hrs) | 0.01 N HCl | pH 6.8 buffer |
|---|---|---|
| 0 | 0 | 0 |
| 1 | 16 | 12 |
| 2 | 24 | 18 |
| 4 | 37 | 29 |
| 8 | 57 | 47 |
| 12 | 72 | 60 |
| 16 | 88 | 74 |
| 20 | 93 | 83 |
| 24 | 98 | 90 |

The results show a slow and sustained release profile for the period of 24 hours. The results also indicate that the release of alfuzosin from the tablets was not significantly affected by the pH of the dissolution medium.

Following are formulation tables showing the compositions of six additional alfuzosin dosage forms. The dosage forms were prepared using the process described above with respect to Example 1.

### Example 2

TABLE 3
Formulation of Example 2

| S. No. | Ingredient | mg/tab | % w/w |
|---|---|---|---|
| 1 | Alfuzosin Hydrochloride | 10 | 2.86 |
| 2 | Hydroxypropyl methylcellulose | 150 | 42.86 |
| 3 | Hydroxypropyl cellulose | 115 | 32.86 |
| 4 | Lactose | 52 | 14.86 |
| 5 | Polyvinyl pyrrolidone (PVP K30) | 15 | 4.29 |
| 6 | Talc | 2 | 0.57 |
| 7 | Colloidal silicon dioxide | 2 | 0.57 |
| 8 | Magnesium stearate | 2 + 2 | 1.14 |
| | Total Core Tablet Weight | 350 | |
| | Coating OPADRY White | 2.5% | |
| | (non-functional dressing) | | |

### Example 3

TABLE 4
Formulation of Example 3

| S. No. | Ingredient | mg/tab | % w/w |
|---|---|---|---|
| 1 | Alfuzosin Hydrochloride | 10 | 2.86 |
| 2 | Hydroxypropyl methylcellulose | 125 | 35.71 |
| 3 | Hydroxypropyl cellulose | 115 | 32.86 |
| 4 | Lactose | 77 | 22.0 |
| 5 | Polyvinyl pyrrolidone | 15 | 4.29 |
| 6 | Talc | 2 | 0.57 |
| 7 | Colloidal silicon dioxide | 2 | 0.57 |
| 8 | Magnesium stearate | 2 + 2 | 1.14 |
| | Total Core Tablet Wt | 350 | |
| | Coating OPADRY White | 2.5% | |
| | (non-functional dressing) | | |

### Example 4

TABLE 5
Formulation of Example 4

| S. No. | Ingredient | mg/tab | % w/w |
|---|---|---|---|
| 1 | Alfuzosin Hydrochloride | 10 | 2.86 |
| 2 | Hydroxypropyl methyl cellulose | 125 | 35.71 |
| | (Methocel K15M CR) | | |
| 3 | Hydroxypropyl cellulose (M) | 115 | 32.86 |
| 4 | Lactose | 77 | 22.0 |
| 5 | Polyvinyl pyrrolidone | 15 | 4.29 |
| 6 | Talc | 2 | 0.57 |
| 7 | Colloidal silicon dioxide | 2 | 0.57 |
| 8 | Magnesium stearate | 2 + 2 | 1.14 |
| | Total Core Tablet Wt | 350 | |
| | Coating OPADRY White | 2.5% | |
| | (non-functional dressing) | | |

### Example 5

TABLE 6
Formulation of Example 5

| S No. | Ingredient | mg/tab | % w/w |
|---|---|---|---|
| 1 | Alfuzosin Hydrochloride | 10 | 3.64 |
| 2 | Hydroxypropyl methyl cellulose | 50 | 18.18 |
| 3 | Hydroxypropyl cellulose (M) | 115 | 41.81 |
| 4 | Lactose | 77 | 28.0 |
| 5 | Polyvinyl pyrrolidone | 15 | 5.45 |
| 6 | Talc | 2 | 0.73 |
| 7 | Colloidal silicon dioxide | 2 | 0.73 |
| 8 | Magnesium stearate | 4 | 1.46 |
| | Total Core Tablet Wt | 275 | |
| | Coating OPADRY White | 2.5% | |
| | (non-functional dressing) | | |

### Example 6

TABLE 7
Formulation of Example 6

| S. No. | Ingredient | mg/tab | % w/w |
|---|---|---|---|
| 1 | Alfuzosin Hydrochloride | 10 | 2.86 |
| 2 | Hydroxypropyl methylcellulose | 80 | 22.86 |
| 3 | Hydroxypropyl cellulose | 75 | 21.43 |
| 4 | Lactose | 162 | 46.29 |
| 5 | Polyvinyl pyrrolidone | 15 | 4.29 |
| 6 | Talc | 2 | 0.57 |
| 7 | Colloidal silicon dioxide | 2 | 0.57 |
| 8 | Magnesium stearate | 2 + 2 | 1.14 |
| | Total Core Tablet Wt | 350 | |

### Example 7

TABLE 8
Formulation of Example 7

| S. No. | Ingredient | mg/tab | % w/w |
|---|---|---|---|
| 1 | Alfuzosin Hydrochloride | 10 | 2.86 |
| 2 | Hydroxypropyl methylcellulose | 100 | 28.57 |
| 3 | Hydroxypropyl cellulose | 95 | 27.14 |
| 4 | Lactose | 122 | 35.86 |
| 5 | Polyvinyl pyrrolidone | 15 | 4.29 |
| 6 | Talc | 2 | 0.57 |
| 7 | Colloidal silicon dioxide | 2 | 0.57 |
| 8 | Magnesium stearate | 2 + 2 | 1.14 |
| | Total Core Tablet wt | 350 | |

Table 9 contains the results of a study of the drug release profile of the tablets of Example 7 in pH 6.8 phosphate buffer using a USP Type II apparatus with a paddle speed of 100 rpm, at 37±2° C. The results indicate that alfuzosin is released in a slow and sustained manner for the period of 24 hours.

TABLE 9
Dissolution Profile of the Formulation of Example 7

| Time (hrs) | Percent released |
|---|---|
| 0 | 0 |
| 1 | 12 |
| 2 | 18 |
| 4 | 29 |
| 8 | 49 |
| 12 | 65 |
| 16 | 78 |
| 20 | 88 |
| 24 | 95 |

### Example 8

TABLE 10
Formulation of Example 8

| S. No. | Ingredient | mg/tab | % w/w |
|---|---|---|---|
| 1 | Alfuzosin Hydrochloride | 10 | 2.78 |
| 2 | Hydroxypropyl methyl cellulose | 80 | 22.22 |
| 3 | Hydroxypropyl cellulose | 75 | 20.83 |
| 4 | Eudragit L-100 55 | 25 | 6.94 |
| 5 | Lactose | 150 | 41.67 |
| 6 | Magnesium Stearate | 3 | 0.83 |
| 7 | Colloidal silicon dioxide | 2 | 0.56 |
| 8 | Polyvinyl pyrrolidone | 15 | 4.17 |
| | Total Core Tablet Weight | 360 | |
| | Coating OPADRY White | 1.5% | |
| | (non-functional dressing) | | |

Alfuzosin and all the other ingredients were initially sifted to break lumps and remove extraneous materials. Alfuzosin then was mixed with colloidal silicon dioxide and geometrically diluted with other excipients, namely, hydroxypropyl methylcellulose, hydroxypropyl cellulose, polyvinyl pyrrolidone, lactose, and Eudragit. The above mixture was lubricated with magnesium stearate and directly compressed into tablets using round punches of 8.5 mm biconvex toolings. The resulting tablets were then coated using an OPADRY dispersion in water. Table 11 shows the results of a study of the drug release profile of these tablets in 0.01N HCl using a USP Type II apparatus with a paddle speed of 100 rpm, 37±2° C. The results indicate that alfuzosin is released in a slow and sustained manner for the period of 24 hours.

TABLE 11
Dissolution Profile of the Formulation of Example 8

| Time (hrs) | Percent released |
|---|---|
| 0 | 0 |
| 1 | 17 |
| 2 | 26 |
| 4 | 39 |
| 8 | 61 |
| 12 | 77 |
| 16 | 88 |
| 20 | 94 |
| 24 | 96 |

### Example 9

TABLE 12
Formulation of Example 9

| S. No. | Ingredients | A Mg | % w/w | B mg | % w/w | C mg | % w/w | D mg | % w/w |
|---|---|---|---|---|---|---|---|---|---|
| 1 | Alfuzosin hydrochloride | 10 | 2.78 | 10 | 3.57 | 10 | 3.51 | 5 | 1.39 |
| 2 | Hydroxypropyl methylcellulose | 80 | 22.22 | 0 | 0 | 80 | 28.07 | 80 | 22.22 |
| | K100MCR | | | | | | | | |
| 3 | Hydroxy propyl cellulose | 75 | 20.83 | 75 | 26.79 | 0 | 0 | 75 | 20.83 |
| 4 | Eudragit L100 55 | 25 | 6.94 | 25 | 8.93 | 25 | 8.77 | 25 | 6.94 |
| 5 | Lactose monohydrate | 150 | 41.67 | 150 | 53.57 | 150 | 52.63 | 155 | 43.06 |
| 7 | Polyvinylpyrrolidone K30D | 15 | 4.17 | 15 | 5.36 | 15 | 5.26 | 15 | 4.17 |
| 8 | Colloidal silicon dioxide | 2 | 0.56 | 2 | 0.71 | 2 | 0.70 | 2 | 0.56 |
| 9 | Magnesium stearate | 3 | 0.83 | 3 | 1.07 | 3 | 1.05 | 3 | 0.83 |
| | Total core | 360 | | 280 | | 285 | | 360 | |

Process:
For Batch no A:

All the ingredients except magnesium stearate and polyvinylpyrrolidone were added to a rapid mixer granulator and blended for 10 minutes. To this blend a 20% w/w solution of polyvinylpyrrolidone in isopropyl alcohol was added to prepare granules. The granules thus obtained were dried in a fluidized bed dryer at 45° C. until the loss on drying is not more than 4% w/w on an IR balance at 105° C. The dried granules were sifted and passed through BSS mesh No 22 and lubricated with magnesium stearate. The lubricated material obtained then was compressed into tablets using round 8.5 mm punches.

For Batch no B, C and D:

All the ingredients were blended by geometric dilution of the drug, lubricated, and directly compressed to tablets using round 8.5 mm punches. The above formulations also may be prepared by wet granulation process.

The compositions according to the various embodiment of the invention may be formulated to produce formulations that are bioequivalent to the 5 mg and 10 mg extended release formulations of alfuzosin prepared using Geomatrix technology. A representative 10 mg extended release composition, made according to the formulation of Example 1 (Table 1), shows the following pharmacokinetic profile.

TABLE 13
Mean (geometric) pharmacokinetic parameters of 10
mg alfuzosin tablets according to Example 1 (Table
1) and that of marketed formulation Xatral.

| | 10 mg SR tablet of | 10 mg ER | Ratio of A/B |
|---|---|---|---|
| | Example 1 (Table 1) | tablet, | 90% Confidence Interval |
| Parameters | (A) | Xatral (B) | [lower limit-upper limit] |
| Cₘₐₓ (ng/ml) | 11.26 | 11.41 | 98.68 |
| | | | [84.97-114.68] |
| Tₘₐₓ (h) | 8.19 | 7.64 | — |
| AUC₀₋₂₄ | 157.21 | 144.66 | 108.67 |
| (ng · h/ml) | | | |
| AUC_{0-inf} | 176.89 | 169.75 | 104.08 |
| (ng · h/ml) | | | [93.29-116.39] |

N = 12 (Healthy male subjects)

A randomized, two treatment, two sequence, single dose, bioavailability study was performed on sustained release Alfuzosin Hydrochloride 10 mg tablets of the present invention versus Xatral-XL 10 mg (Sanofi Synthelabo, product from UK market) under fed conditions (U.S. F.D.A. standard meal) on 12 healthy human volunteers. As shown in Table 13, the 10 mg compositions of alfuzosin prepared according to the formulation of Example 1 (Table 1) were found to be bioequivalent to the reference product, 10 mg Xatral-XL marketed in European countries. It is believed that the compositions may also prove to be bioequivalent to 10 mg UroXatral tablets, as and when these tablets become available in the USA. Compositions that are bioequivalent to 5 mg and 10 mg extended release alfuzosin tablets available in other countries may be formulated accordingly. Such modified compositions are contemplated to be within the scope of the appended claims.

While several particular forms of the inventions have been described, it will be apparent that various modifications and combinations of the inventions detailed in the text can be made without departing from the spirit and scope of the inventions. Further, it is contemplated that any single feature or any combination of optional features of the inventive variations described herein may be specifically excluded from the claimed inventions and be so described as a negative limitation. Accordingly, it is not intended that the inventions be limited, except as by the appended claims.

We claim:

1. A sustained release oral dosage form comprising:
a single functional layer; and
optionally, one or more nonfunctional layers adjacent to the single functional layer,
wherein the single functional layer comprises alfuzosin or pharmaceutically acceptable salt, solvate, enantiomers or mixtures thereof and one or more release retarding ingredients.

2. The sustained release oral dosage form of claim 1, wherein the release retarding ingredient comprises one or more of cellulose polymer, methacrylate polymer, acrylic acid polymer, block copolymer, gum and polyethylene oxide.

3. The sustained release oral dosage form of claim 2, wherein the cellulose polymer comprises one or more of hydroxypropyl methylcellulose, methylcellulose, hydroxypropylethylcellulose and hydroxypropyl cellulose.

4. The sustained release oral dosage form of claim 2, wherein the gum comprises one or more of xanthan gum, alginic acid, sodium alginate and locust bean gum.

5. The sustained release dosage form of claim 1, wherein the single functional layer further comprises one or more pharmaceutically acceptable excipients.

6. The sustained release oral dosage form of claim 1, wherein the one or more pharmaceutically acceptable excipients comprise one or more of binders, diluents, and lubricants/glidants.

7. The sustained release oral dosage form of claim 6, wherein the binders comprise one or more of polyvinyl pyrrolidone, pregelatinized starch, and gelatin.

8. The sustained release oral dosage form of claim 6, wherein the diluents comprise one or more of lactose, mannitol, and microcrystalline cellulose.

9. The sustained release oral dosage form of claim 6, wherein the lubricants comprise one or more of magnesium stearate, zinc stearate, talc, and colloidal silicon dioxide.

10. The sustained release oral dosage form of claim 1, wherein the functional layer comprises between about 10% to about 90% w/w of hydroxypropyl methylcellulose and between about 10% to about 90% w/w of hydroxypropyl cellulose.

11. The sustained release oral dosage form of claim 1, wherein the functional layer comprises between about 10% to about 70% w/w of hydroxypropyl methylcellulose, between about 10% to about 70% w/w of hydroxypropyl cellulose and between about 1% to about 20% w/w of methacrylic acid copolymer.

12. The sustained release oral dosage form of claim 1, wherein the functional layer comprises between about 10% to about 70% w/w of hydroxypropyl methylcellulose, between about 10% to about 70% w/w of hydroxypropyl cellulose, between about 5% to about 10% w/w of methacrylic acid copolymer, and between about 10% to about 50% w/w of lactose.

13. The sustained release dosage form of claim 1, wherein the dosage form comprises one or more of tablets, capsules, pellets, granules and other dosage forms suitable for oral administration.

14. The sustained release oral dosage form of claim 1, wherein the dosage form has a dissolution of less than about 17% in about 1 hour, less than about 61% in about 8 hours, less than about 94% in about 20 hours, as measured in a pH 6.8 phosphate buffer using USP Type II apparatus with a paddle speed of 100 rpm, at 37±2° C.

15. The sustained release oral dosage form of claim 1, wherein the dosage form has a dissolution of less than about 26% in about 2 hours, less than about 77% in about 12 hours, and less than about 96% in about 24 hours, as measured in a pH 6.8 phosphate buffer using USP Type II apparatus with a paddle speed of 100 rpm, at 37±2° C.

16. The sustained release oral dosage form of claim 1, wherein the dosage form has a dissolution of less than about 39% in about 4 hours and less than about 88% in about 16 hours, as measured in a pH 6.8 phosphate buffer using USP Type II apparatus with a paddle speed of 100 rpm, at 37±2° C.

17. The sustained release oral dosage form of claim 1, wherein the single functional layer comprises granules.

18. The sustained release oral dosage form of claim 1, wherein the one or more nonfunctional layers adjacent to the single functional layer comprises a cosmetic coating.

19. The sustained release oral dosage form of claim 18, wherein the cosmetic coating comprises a colorant.

20. A method of treating secondary symptoms associated with benign prostatic hyperplasia, the method of treating comprising administering a sustained release oral dosage form, the sustained release oral dosage form comprising a single functional layer and, optionally, one or more nonfunctional layers adjacent to the single functional layer,
wherein the single functional layer comprises alfuzosin or pharmaceutically acceptable salt, solvate, enantiomers or mixtures thereof and one or more release retarding ingredients.

21. The method of claim 20, wherein the release retarding ingredient comprises one or more of cellulose polymer, methacrylate polymer, acrylic acid polymer, block copolymer, gum and polyethylene oxide.

22. The method of claim 21, wherein the cellulose polymer comprises one or more of hydroxypropyl methylcellulose, methylcellulose, hydroxypropylethylcellulose and hydroxypropyl cellulose.

23. The method of claim 21, wherein the gum comprises one or more of xanthan gum, alginic acid, sodium alginate and locust bean gum.

24. The method of claim 20, wherein the sustained release oral dosage form is administered twice daily.

25. The method of claim 20, wherein the sustained release oral dosage form is administered once daily.

26. The method of claim 20, wherein the single functional layer comprises granules.

27. The method of claim 20, wherein the one or more nonfunctional layers adjacent to the single functional layer comprise a cosmetic coating.

28. The method of claim 27, wherein the cosmetic coating comprises a colorant.

29. A process for forming a sustained release oral dosage form, the process comprising:
forming a mixture of alfuzosin or pharmaceutically acceptable salt, solvate, enantiomers or mixtures thereof and one or more release retarding ingredients;
forming a dosage form having a single functional layer from the mixture; and
optionally forming one or more nonfunctional layers adjacent to the single functional layer.

30. The process of claim 29, wherein the release retarding ingredient comprises one or more of cellulose polymer, methacrylate polymer, acrylic acid polymer, block copolymer, gum and polyethylene oxide.

31. The process of claim 30, wherein the cellulose polymer comprises one or more of hydroxypropyl methylcellulose, methylcellulose, hydroxypropyl ethylcellulose and hydroxypropyl cellulose.

32. The process of claim 30, wherein the gum comprises one or more of xanthan gum, alginic acid, sodium alginate and locust bean gum.

33. The process of claim 29, wherein the one or more nonfunctional layers adjacent to the single functional layer comprise a cosmetic coating.

34. The process of claim 33, wherein the cosmetic coating comprises a colorant.

35. The process of claim 29, wherein forming a mixture comprises one or more of direct compression, wet granulation, and dry granulation.

36. The process of claim 29, wherein forming a mixture comprises forming granules that comprise the alfuzosin or pharmaceutically acceptable salt, solvate, enantiomers or mixtures thereof and one or more release retarding ingredients.

37. The process of claim 29, wherein forming a mixture further comprises adding one or more pharmaceutically acceptable excipients to the mixture.

38. The process of claim 29, wherein forming the dosage form having a single functional layer comprises one or more of forming tablets, capsules, pellets, granules or other dosage form suitable for oral administration.

39. The process of claim 38, wherein forming the dosage form further comprises compressing to form a tablet.

40. The process of claim 38, wherein forming the dosage form further comprises filling into a capsule.
